(19) United States
(12) Patent Application Publication
(10) Pub. No.: US 2026/0096966 A1
(43) Pub. Date: Apr. 9, 2026

(54) Compositions Containing a UV-Absorbing System Including Physical UV-Attenuating Material and An Emollient System

(71) Applicant: L'OREAL, PARIS (FR)

(72) Inventors: BRIAN BODNAR, MANASQUAN, NJ (US); ANIL SHAH, EAST WINDSOR, NJ (US)

(73) Assignee: L'OREAL, PARIS (FR)

(21) Appl. No.: 19/349,244

(22) Filed: Oct. 3, 2025

Related U.S. Application Data
(60) Provisional application No. 63/703,336, filed on Oct. 4, 2024.

(30) Foreign Application Priority Data
Dec. 10, 2024 (FR) 2413782

(51) Int. Cl. A61K 8/27; A61K 8/02; A61K 8/06; A61K 8/25; A61K 8/26; A61K 8/29; A61K 8/31; A61K 8/365; A61K 8/37; A61K 8/891; A61Q 17/04

(52) U.S. Cl. CPC A61K 8/27; A61K 8/0241; A61K 8/06; A61K 8/25; A61K 8/26; A61K 8/29; A61K 8/31; A61K 8/365; A61K 8/37; A61K 8/891; A61Q 17/04; A61K 2800/30; A61K 2800/413; A61K 2800/621; A61K 2800/622; A61K 2800/624; A61K 2800/651

(57) ABSTRACT

The present disclosure relates to compositions including a UV absorbing system including at least one physical ultraviolet (UV)-attenuating material and emollient system comprising at least three emollients, as well as to methods of making and using such compositions.

### CROSS-REFERENCE TO RELATED APPLICATIONS

The present application claims priority to U.S. Provisional Patent Application No. 63/703,336 filed Oct. 4, 2024 and French patent application No. FR 2413782 filed on Dec. 10, 2024. The entire contents of the aforementioned patent applications are incorporated herein by this reference.

### FIELD OF THE INVENTION

The present disclosure relates to compositions comprising a UV absorbing system comprising at least one physical ultraviolet (UV)-attenuating material and an emollient system comprising at least three emollients, as well as to methods of making and using such compositions.

### DISCUSSION OF BACKGROUND

Exposure to radiation of wavelengths between 290 nm and 400 nm, called UV radiation or simply “UVR”, is implicated in a number of skin-related diseases and conditions. Exposure to radiation of wavelengths between 290 and 320 nm, also called UVB radiation, can lead to many detrimental changes in the biomechanical properties, appearance, and health of the epidermis, resulting in erythema (sunburn), immediate skin darkening, appearance of wrinkles leading to the premature aging of the skin (i.e., photoaging), and a variety of other skin health-related concerns which may include melanoma or other skin photocancers.

UVA rays with wavelengths between 320 and 400 nm penetrate deeper into the skin than UVB rays. Exposure to UVA rays may cause immediate and persistent browning of the skin, and other changes to the texture or appearance of the skin. Daily exposure to UVA rays, even for a short time, under normal conditions can damage collagen fibers and elastin, resulting in a change in the microrelief of the skin, the appearance of wrinkles and uneven pigmentation (spots, lack of uniformity of complexion).

Many studies show the need for effective protection against UVA and UVB to prevent sunburn, photoaging, and other skin conditions or diseases related to UVR exposure.

In order to obtain a product with high protection against UVR, it is generally necessary to combine a large number of sunscreens and/or a high amount of UV filters to achieve high levels of filtering efficiency.

However, high levels of UV filters do not lend themselves to an easy elaboration of stable compositions with a pleasant texture. It is possible to achieve high UV protection using one or more organic UV filters; however, organic UV filters are often very greasy and unpleasant when applied to the skin, especially when used at high levels in a sunscreen formulation required for high UV protection efficacy.

Mineral UV filters such as titanium dioxide or zinc oxide are alternative UV filter choices to organic UV filters which can bring more pleasant textures; however, when one or more mineral UV filters are used, the resulting sunscreen formulation is far too whitening (also referred to as “white cast”) when applied to the skin. When mineral UV filters are used at lower levels, it may be possible to produce sunscreen formulations with little to no whitening when applied to skin; however, such formulations containing low levels of mineral UV filters are not able to yield desired high levels of UV protection.

Finally, combining organic UV filter and mineral UV filter in a single composition can be difficult, particularly doing so in a manner which results in a non-greasy, pleasant, non-whitening, high UV protection sunscreen formulation, especially considering the need to balance UVA and UVB protection efficacy.

There remains a need in the art for improved sunscreen compositions which possess high UV protection and balanced UVA and UVB protection, which are not greasy, are pleasant to apply, and which are not whitening when applied to the skin.

Accordingly, one aspect of the present disclosure is a composition which is not greasy, which is non-whitening when applied to the skin, and which has high UV protection.

### SUMMARY OF THE INVENTION

The present disclosure relates to compositions comprising a UV absorbing system comprising at least one physical ultraviolet (UV)-attenuating material and an emollient system comprising at least three emollients, wherein at least one emollient is a fast-spreading emollient, at least one emollient is a medium spreading emollient, and at least one emollient is a slow spreading emollient. Preferably, the compositions are non-greasy and/or non-whitening (that is, the compositions do not provide a white cast) upon application.

The present disclosure also relates to methods of treating, caring for, protecting, enhancing the appearance of, and/or making up a keratinous material comprising applying compositions of the present disclosure to a keratinous material in an amount sufficient to treat, care for, enhance the appearance of, and/or make up the keratinous material.

The present disclosure also relates to methods of making non-greasy and/or non-whitening compositions comprising at least one physical ultraviolet (UV)-attenuating material by combining at least one physical ultraviolet (UV)-attenuating material, at least one fast spreading emollient, at least medium spreading emollient, and at least one slow spreading emollient during formation of the compositions to produce compositions which are non-greasy and/or non-whitening.

The present disclosure also relates to methods of improving sensory properties upon application to skin of a composition comprising at least one physical ultraviolet (UV)-attenuating material, wherein the method comprises adding at least one fast spreading emollient, at least medium spreading emollient, and at least one slow spreading emollient to a composition comprising at least one physical ultraviolet (UV)-attenuating material during formation of the composition in amounts sufficient to improve sensory properties of the composition upon application to skin.

It is to be understood that both the foregoing general description and the following detailed description are exemplary and explanatory only, and are not restrictive of the disclosure.

### DETAILED DESCRIPTION OF THE INVENTION

In the following description and the claims appended hereto, it is to be understood that the terms used have their ordinary and accustomed meanings in the art, unless otherwise specified.

“About” as used herein means within 10% of the indicated number (e.g., “about 10%” means 9%-11% and “about 2%” means 1.8%-2.2%).

“A” or “an” as used herein means “at least one.”

“At least one” means one or more and thus includes individual components as well as mixtures/combinations.

As used herein, all ranges provided are meant to include every specific point and range within, and combination of subranges between, the given ranges. Thus, a range from 1-5 includes specifically the integers within the range 1, 2, 3, 4 and 5, as well as subranges such as and 2-5, 3-5, 2-3, 2-4, 1-4, etc., as well as all fractional numbers within the range such as 1.2, 2.3, 3.4, etc., and subranges including such fractional numbers such as 1.5-3.8, 2-4.3, 4.2-4.9, etc.

“Film former”, “film-forming polymer” or “film-forming agent” as used herein means a polymer or resin which is capable of leaving a film on the substrate to which it is applied, for example, after a solvent accompanying the film former has evaporated, absorbed into and/or dissipated on the substrate.

“Substituted” as used herein, means comprising at least one substituent. Non-limiting examples of substituents include atoms, such as hydrogen atoms or chlorine atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 115° C.

“Non-volatile”, as used herein, means having a flash point of greater than about 115° C.

“Polymer” as used herein means a compound which is made up of at least two monomers.

“Fast spreading emollient” as used herein means emollients which glide easily across keratinous material (skin) and quickly absorb after application, and do not form visibly noticeable film on keratinous material (skin) after application. “Fast spreading emollients” have a low molecular weight (that is, less than about 250 g/mol). Preferably, they also have a low viscosity of less than about 10 mPa-s at or around ambient temperature (25° C.).

“Slow spreading emollient” as used herein means emollients which spread slowly and form a visibly noticeable film on keratinous material (skin) after application. “Slow spreading emollients” may also feel “heavy” or “more substantial” that other emollients. “Slow spreading emollients” have a high molecular weight (that is, greater than about 300 g/mol). Preferably, they also have a high viscosity of greater than about 10 mPa-s at or around ambient temperature (25° C.).

“Medium spreading emollient” as used herein means emollients falling between “fast spreading emollients” and “slow spreading emollients.” “Medium spreading emollients” have a medium molecular weight (that is, between about 250 g/mol and about 300 g/mol) and do not have a preferential viscosity.

“Free” or “substantially free” or “devoid of” as it is used herein means that while it is preferred that no amount of the specific component be present in the composition, it is possible to have very small amounts of it in the compositions of the disclosure provided that these amounts do not materially affect at least one, preferably most, of the advantageous properties of the compositions of the disclosure. Thus, for example, “free of colorant” means that an effective amount (that is, more than trace amounts) of colorant(s) is/are omitted from the composition (that is, about 0% by weight), “substantially free of colorant” means that colorant(s) is/are present in amounts not greater than 0.1% by weight, and “devoid of colorant” means that colorant(s) is/are present in amounts not greater than 0.25% by weight, based on the total weight of the composition. The same nomenclature applies for all other ingredients identified throughout the application and in this paragraph such as, for example, specific UV filters and/or surfactants (compositions of the disclosure which are “free of oxybenzone and/or octinoxate,” “substantially free of oxybenzone and/or octinoxate,” and “devoid of oxybenzone and/or octinoxate,” as well as “free of surfactants,” “substantially free of surfactants,” and “devoid of surfactants,” have meanings consistent with the discussion within this paragraph), even if not specifically discussed for each identified ingredient in the application. Discussed examples of the use of such language such as those in this paragraph are intended to be exemplary, not limiting.

“UV filters” as it is used herein means sunscreen active agents approved by a governmental regulatory agency such as the Food and Drug Administration (FDA) in the U.S. or the EU Commission in Europe and includes organic UV filters such as avobenzone, octocrylene, benzophenones, benzotriazoles and merocyanines, as well as physical ultraviolet (UV)-attenuating materials.

“Whitening” or “white cast” as used herein refers to the visually white appearance of keratinous substance after a composition has been applied to the keratinous material as compared to the appearance of the keratinous material prior to application of the composition. A composition is “non-whitening” if it provides minimal or no visually white appearance (preferably no visually white appearance) to keratinous material upon application.

“Anhydrous” as it is used herein means that compositions of the disclosure contain less than 3% water, meaning that the compositions can also contain less than 2% water, and less than 1% water, as well as being “free of water,” “substantially free of water,” and “devoid of water” as defined above.

A “UV absorbing system essentially containing at least one physical ultraviolet (UV)-attenuating material” as it is used herein means that compositions of the disclosure contain less than 3% UV filters other than physical ultraviolet (UV)-attenuating material(s), in particular less than 3% of UV organic filters, meaning that within this definition as subcategories are compositions containing less than 2% UV filters other than physical ultraviolet (UV)-attenuating material(s), in particular less than 2% of UV organic filters, and less than 1% UV filters other than physical ultraviolet (UV)-attenuating material(s), in particular less than 1% of UV organic filters, as well as being “free of UV filters other than physical ultraviolet (UV)-attenuating material,” “substantially free of UV filters other than physical ultraviolet (UV)-attenuating material,” and “devoid of UV filters other than physical ultraviolet (UV)-attenuating material” as defined above.

“System” and “component” are used interchangeably in this application.

“Primary Particle” as used in connection with the description of physical ultraviolet (UV)-attenuating material herein means inorganic or organic particles (structures) which can be held together via molecular or atomic bonding to form a physical ultraviolet (UV)-attenuating material.

“Primary particle size” means the size of an unaggregated primary particle in a physical ultraviolet (UV)-attenuating material.

“Passivated” as used in connection with the description of physical ultraviolet (UV)-attenuating material herein refers to a material which has been treated in a way such that the potential to release ionic species when in contact with water is reduced compared to the same non-passivated material.

“Keratinous materials” or “keratinous substance” means nails (finger and/or toe nails), skin such as body, face, and eye area, scalp, keratin fibers such as eyelashes, eyebrows, and hair, and mucous membranes such as lips.

“Physiologically acceptable” means compatible with keratinous materials and having a pleasant color, odor and feel, and which does not cause any unacceptable discomfort (stinging or tautness) liable to discourage a consumer from using the composition.

“UV protection efficiency” or “filtering efficiency” in the context of the present disclosure, is evaluated from one or more of SPF, UVAPF, Critical Wavelength, and UVA-I/UV ratio.

“SPF” (Sun Protection Factor) measures the level of protection against erythema provided by a composition. The SPF value corresponds to the ratio between the minimum erythemal dosage (MED) measured wearing the composition and the MED measured with bare skin. “SPF” is a known term in the sunscreen art and is defined, for example, in A new substrate to measure sunscreen protection factors throughout the ultraviolet spectrum, J. Soc. Cosmet. Chem., 40, 127-133 (May/June 1989).

The evaluation of the SPF (Sun Protection Factor) can be performed, for example, in vitro with spectrophotometer by Labsphere (North Sutton, NH, USA). In such an evaluation, the plate is the material on which the tested composition is applied. For such an evaluation, polymethylmethacrylate (PMMA) plates can be used. An example of an acceptable protocol is in the process of ISO accreditation under the name ISO Committee Draft 23675.

The evaluation of the Sun Protection Factor (SPF) can also be performed in-vivo according to the ISO 24444:2019 protocol “Cosmetics-Sun protection test methods-In-vivo determination of the sun protection factor (SPF).” Also, it can be determined according to FDA protocols, as described in the document “Labeling and Effectiveness Testing; Sunscreen Drug Products for Over-the-Counter Human Use” published in the US Federal Register on Jul. 5, 2011 (https://www.federalregister.gov/d/2011-14766); 21 C.F.R. Part 352 Subpart D § 352.72, updated and revised by the 2011 publication in the Federal Register.

“UVAPF” (UVA protection factor) relates to an index characterizing the protection against UVA provided by a composition. For example, the UVAPF index can be measured in vivo according to the “PPD” (Persistent Pigment Darkening) method in the ISO-24442:2022 protocol, measuring observed skin color 2 to 4 hours after UVA exposure. Also, it can be determined according to FDA protocols, again as described in 21 C.F.R. Part 352 Subpart D § 352.72 as discussed above in connection with SPF.

The evaluation of UVA protection can also be measured in vitro with the Labsphere® spectrophotometer under conditions such as those discussed above in connection with SPF. ISO 24443:2021 protocol describes such an in vitro method.

FDA broad spectrum testing procedures, in particular “critical wavelength” testing procedures, can also be found at 21 C.F.R. Part 352 Subpart D § 352.72. Also, broad spectrum testing procedures include determining the UVA1/UV ratio as described in “Sunscreen Drug Products for Over-the-Counter Human Use” published in the Federal Register https://www.federalregister.gov/documents/2019 Feb. 26/2019-03019/sunscreen-drug-products-for-over-the-counter-human-use.

According to the present disclosure, compositions of the present disclosure preferably have one or more of the following properties:
Compositions have a critical wavelength as determined by FDA's critical wavelength procedures of at least 370 nm;
Compositions have an SPF value of at least 15, preferably at least 30, preferably at least 50 and preferably at least 70;
Compositions have a UVAPF/SPF ratio of at least ⅓, and preferably at least ⅖; and/or
Compositions have a UVA1/UV ratio of 0.7 or higher, preferably 0.75 or higher, and preferably 0.8 or higher.

“Makeup Result” as used herein, refers to compositions where color remains the same or substantially the same as at the time of application, as viewed by the naked eye, after an extended period of time. “Makeup Result” may be evaluated by evaluating long wear properties by any method known in the art for evaluating such properties. For example, long wear may be evaluated by a test involving the application of a composition to keratinous material such as skin and evaluating the color of the composition after an extended period of time. For example, the color of a composition may be evaluated immediately following application to keratinous material such as skin and these characteristics may then be re-evaluated and compared after a certain amount of time. Further, these characteristics may be evaluated with respect to other compositions, such as commercially available compositions.

“Natural” as in the phrase “natural compound” refers to any compound derived directly from a natural substance such as a plant without having undergone any chemical modification.

“Compound of natural origin” refers to any compound derived from a natural compound which has undergone one or more chemical modifications, for example by organic synthesis reaction, without the properties of the natural compound having been modified.

“Synthetic compound” refers to any compound which is not a natural compound or a compound of natural origin.

“Room temperature” means about 20-25° C.

“Atmospheric pressure” means about 760 mmHg, i.e. about 105 pascals.

“UV filter” and “sunscreen agent” are used interchangeably in this application.

“UV efficacy” and “UV efficiency” and “UV protection efficacy” are used interchangeably in this application.

The compositions and methods of the present disclosure can comprise, consist of, or consist essentially of the essential elements and limitations of the disclosure described herein, as well as any additional or optional ingredients, components, or limitations described herein or otherwise useful. For example, the UV (ultraviolet) absorbing system of the compositions of the disclosure can “consist essentially of” at least one physical ultraviolet (UV)-attenuating material(s).

For purposes of the present disclosure, the “basic and novel property” associated with compositions, components and methods related to UV protection properties which “consist essentially of” identified ingredients or actions is “UVA protection indicated by UVAPF/SPF and UVA1/UV ratios.”

For purposes of the present disclosure, the “basic and novel property” associated with compositions, components and methods related to composition properties upon application to keratinous material “consist essentially of” identified ingredients or actions is “improved sensory properties upon application to skin.”

“Improving sensory properties upon application to skin” as used herein refers to spreading sensory properties upon application to skin over time during application. The most optimal “sensory properties” as used herein refer to compositions which have a pleasing, gliding and lubricious feel throughout the application of the product to a keratinous substrate. By way of comparative example, using only fast spreading emollients in a composition would result in a composition which had a pleasant, lubricious feel only on initial application, and would become unpleasant throughout the remaining time of application. Conversely, using only slow spreading emollients in a composition would result in a composition which would feel heavy and greasy throughout application. Using only medium spreading emollients would result in a composition which would not feel especially pleasant or lubricious throughout application.

Compositions of the present disclosure may be in any form suitable for use as a personal care composition, such as that of a stick, a paste, a cream, an anhydrous composition, an emulsion (oil-in-water, water-in-oil, multiple emulsion such as oil-in-water-in-oil), nanoemulsion, a gel, a liquid, a solid, etc. These compositions can be used for any personal care purpose in cosmetic and/or dermatological products such as, for example, a sunscreen, a foundation, lip balms, lipsticks, concealers, mascaras, leave-in hair products, eye shadows, powders, etc.

Referred to herein are trade names for materials including, but not limited to, materials such as polymers and optional components. Materials are not intended to be limited by materials described and referenced by a certain trade name herein. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

All U.S. patents or patent applications disclosed herein are expressly incorporated by reference in their entirety.

### Composition

### Physical UV-Attenuating Material

According to the present disclosure, compositions comprising at least one physical ultraviolet (UV)-attenuating material are provided. “Physical ultraviolet (UV)-attenuating material” as used herein refers to solid inorganic ingredients which absorb incoming ultraviolet (UV)-light, and may optionally scatter incoming ultraviolet (UV)-light, when present in compositions disclosed herein. The physical UV-attenuating material preferably comprises metal oxide(s) such as, for example, oxides of titanium, chromium, zinc, tin, alumina, cerium, and/or iron. Specific examples of suitable metal oxide(s) include, but are not limited to, at least one metal oxide selected from the group consisting of titanium dioxide, zinc oxide, iron oxide, chromium oxide, tin oxide, alumina, cerium oxide, and mixtures thereof.

The physical UV-attenuating material may be subjected to a surface treatment agent to improve sensory, performance, and/or compatibility of the compositions disclosed herein. Suitable surface treatment agents may include hydrophobic or hydrophilic surface treatment agents such as, for example, those described in Cosmetics & Toiletries, February 1990, Vol. 105, pp. 53-64, including but not limited to specific examples such as amino acids, beeswax, fatty acids, fatty acid salts, fatty alcohols, anionic surfactants, lecithin, lecithin derivatives, metal alkoxides, polyethylene, silicones, proteins, alkanolamines, silicon oxides, metal oxides, sodium hexametaphosphate, alumina, and/or glycerol. Preferably, the physical UV-attenuating material is passivated.

Preferably, the physical UV-attenuating material comprises one or more of zinc oxide and/or titanium dioxide. Preferably, the physical UV-attenuating material comprising one or more of zinc oxide and/or titanium dioxide is passivated.

According to preferred embodiments, the physical UV-attenuating material comprises titanium dioxide. Titanium dioxide may be present in any form in the compositions disclosed herein. Further, the TiO2 can be treated (coated) or untreated.

According to preferred embodiments, the physical UV-attenuating material comprises zinc oxide. Zinc oxide may be present in any form (e.g., wurtzite or zincblende form) in the compositions disclosed herein. Further, the zinc oxide can be treated (coated) or untreated.

Preferably, the physical UV-attenuating material comprises zinc oxide. Preferably, the zinc oxide is passivated.

Preferably, the physical UV-attenuating material comprises titanium dioxide. Preferably, the titanium dioxide is passivated.

Preferably, mean primary particle size of the physical ultraviolet (UV)-attenuating material is from 1 nm to 500 nm, preferably from 5 nm to 250 nm, preferably from 10 nm to 100 nm, and preferably from 20 nm to 50 nm, including all ranges and subranges therebetween such as, for example, 25 nm to 40 nm, 10 nm to 75 nm, and 15 nm to 150 nm.

Suitable examples of coated pigments include but are not limited to titanium dioxides that have been coated such as titanium dioxides:
with hydrated silica, such as the product MT-100WP from Tayca,
with silica and iron oxide, such as the product Sunveil FR from Ikeda,
with silica and alumina, such as the products MT-500SA® and MT-100SA® from Tayca and Tioveil™ AQ-N from Croda,
with alumina, such as the product TTO-55 (A)® from Ishihara,
with alumina and aluminium stearate, such as the products MT-100TV®, MT-100Z® and MT-01® from Tayca, the product Solaveil™ CT100® from Croda and the product Eusolex T-AVO® from Merck,
with silica, alumina and alginic acid, such as the product MT-100AQ® from Tayca,
with alumina and aluminium laurate,
with iron oxide and iron stearate,
with zinc oxide and zinc stearate,
with silica and alumina and treated with a silicone, such as the products MTY-500SAS® or Microtitanium Dioxide MT-100SAS® from Tayca,
with silica, alumina, and aluminium stearate and treated with a silicone,
with silica and treated with a silicone,
with silica and treated with a silicone, such as the product TTO-55 (S)® from Ishihara,
with triethanolamine,
with stearic acid, such as the product TTO-55 (C)® from Ishihara,
with sodium hexametaphosphate,
with octyltrimethylsilane,
with a polydimethylsiloxane, with anatase/rutile TiO₂ treated with a polydimethylhydrogenosiloxane,
with triethylhexanoin, with aluminium stearate and with alumina sold under the trade name Solaveil™ CT-200 by Croda,
with aluminium stearate, with alumina and with silicone, sold under the trade name Solaveil™ CT-12W by Croda,
with lauroyl lysine, and/or
with C₉-C₁₅ fluoroalcohol phosphate and with aluminium hydroxide.

Mention may also be made of TiO₂ pigments doped with at least one transition metal such as iron, zinc or manganese, preferably manganese. Preferably, the doped pigments are in the form of an oily dispersion. The oil present in the oily dispersion is preferably chosen from triglycerides such as, for example, capric/caprylic acids. The oily dispersion of titanium oxide particles may also comprise one or more dispersants, for instance a sorbitan ester, for instance sorbitan isostearate, or a polyoxyalkylenated fatty acid ester of glycerol, for instance Tri-PPG-3 myristyl ether citrate and polyglyceryl-3 polyricinoleate. Preferably, the oily dispersion of titanium oxide particles includes at least one dispersant chosen from polyoxyalkylenated fatty acid esters of glycerol. Mention may be made more particularly of the oily dispersion of TiO₂ particles doped with manganese in capric/caprylic acid triglyceride in the presence of Tri-PPG-3 myristyl ether citrate and polyglyceryl-3 polyricinoleate and sorbitan isostearate having the INCI name: titanium dioxide (and) TRI-PPG-3 myristyl ether citrate (and) polyglyceryl-3 ricinoleate (and) sorbitan isostearate, for instance the product sold under the trade name Optisol™ OTP-1 by Croda.

Suitable uncoated titanium oxide includes, but it not limited to, those sold by Tayca under the trade names MT-500B or MT-600BR, or by Evonik under the name Degussa P 25.

Suitable examples of uncoated zinc oxide include, but are not limited to, zinc oxide marketed under the name “Z-COTE”® by BASF, zinc oxide marketed under the name “NanoArc® Zinc Oxide” by the company Nanophase Technologies, zinc oxide marketed under the name “MZ-500”, “MZ-300”, “MZ-200” or “MZ-150” by TAYCA.

Treated (coated) zinc oxide compounds are compounds that have undergone one or more surface treatments of chemical, electronic, mechanochemical and/or mechanical nature with compounds as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron or aluminum salts of fatty acids, metal alkoxides (titanium or aluminum), polyethylene, silicones, hydrated silica, proteins (collagen, elastin), alkanolamines, silicon oxides, triethoxycaprylylsilane, metal oxides or sodium hexametaphosphate.

Examples of suitable coated zinc oxide include, but are not limited to, zinc oxide coated with polymethylhydrogensiloxane; zinc oxide dispersed in C12-15 alkyl benzonate (INCI: Zinc Oxide (and) C12-15 Alkyl Benzoate (and) Polyhydroxystearic Acid (and) Isostearic Acid), marketed by Croda under the tradename Sovaveil CZ-100; zinc oxide dispersions in C9-12 alkane with a dispersing agent, marketed under the tradename “DAITOPERSION Zn-60VA”® by the company Daito Kasei; ZnO coated with silicone grafted acrylic polymer, dispersed in cyclodimethylsiloxane, marketed under the name “SPD-Z5®” by Shin-Etsu; ZnO coated with hydrated silica, marketed by TAYCA under the name “MZ-500HP”; ZnO coated with hydrated silica, triethoxysilylethyl polydimethylsiloxyethyl hexyl dimethicone and hydrogen dimethicone (H-Me-Si), marketed by TAYCA under the name MZ-510 HPSX; ZnO coated with stearic acid or isostearic acid, such as those marketed by TAYCA under the name “MZ-505T”, “MZY-505EX” or “MZY-304EX”; ZnO coated with silicone oil, such as those marketed by TAYCA under the name “MZX-510HPS”, “MZY-505S”, “MZY-510M3S”, “MZ-505M”, “MZY-303S”, “MZY-303M”, “MZY-203S”, “MZY-210M3S” or “MZY-153S”; ZnO coated with triethoxycaprylylsilane, such as those sold by BASF under the name Z-COTE HP1, or by TAYCA under the name “MZX-508OTS”, “MZY-203OTS” or “MZX-304OTS” or by DSM under the name PARSOL ZX; for example: ZnO marketed under the trademark “Oxide Zinc CS-5” by Toshiba (ZnO coated with polymethylhydrosiloxane); ZnO marketed under the trademark “Nanogard Zinc Oxide FN” by Nanophase Technologies (as a 40% dispersion in Finsolv TN, C12-C15 alkyl benzoate); ZnO marketed under the trademark “Daitopersion Zn-30” and “Daitopersion Zn-50” by Daito (dispersions in oxyethylenated polydimethylsiloxane/cyclopolymethylsiloxane comprising 30% or 50% of zinc nano-oxides coated with silica and polymethylhydrosiloxane); ZnO marketed under the trademark “NFD Ultrafine ZnO” by Daikin (ZnO coated with phosphate of perfluoroalkyl and a copolymer based on perfluoroalkylethyl as a dispersion in cyclopentasiloxane); ZnO marketed under the trademark “SPD-Z1” by Shin-Etsu (ZnO coated with a silicone-grafted acrylic polymer dispersed in cyclodimethylsiloxane); ZnO marketed under the trademark “Escalol Z100” by ISP (alumina-treated ZnO dispersed in an ethylhexyl methoxycinnamate/PVP-hexadecene copolymer/methicone mixture); ZnO marketed under the trademark “Fuji ZnO-SMS-10” by Fuji Pigment (ZnO coated with silica and polymethylsilsesquioxane); and ZnO marketed under the trademark “Nanox Gel TN” by Elementis (ZnO dispersed at 55% in C12-C15 alkyl benzoate with hydroxystearic acid polycondensate); ZnO marketed under the trademark Finex by SAKAI such as FINEX-50LP, FINEX-50S-LP2 and FINEX-30S-LPT (ZnO coated with Hydrogen dimethicone); FINEX-33W (ZnO coated with Hydrated silica), FINEX-52W-LP2 and FINEX-33W-LP2 (ZnO coated with Hydrogen dimethicone and Hydrated silica), FINEX-50-OTS and FINEX-30-OTS (ZnO coated with Triethoxycaprylysilane).

Preferred coatings for zinc oxide preferably comprise one or more of the following: hydrated silica, triethoxysilylethyl polydimethylsiloxyethyl, hexyl dimethicone, hydrogen dimethicone, and/or triethoxycaprylysilane.

According to preferred embodiments of the present disclosure, the zinc oxide may be in platelet form, and may be coated or uncoated. Suitable examples of such forms are sold by Croda under the Solaveil (MicNo) name such as Solaveil MXP3, MZP7, MZP8, MZ3-100, MZ3-300 AND MZ7-100. Preferably, zinc oxide platelets useful according to the present disclosure (1) have a median specific surface area of more than 25 square meters per gram, preferably greater than 30 square meters per gram, and/or (2) are transparent (that is, >30% transmission at 600 nm). Suitable examples of such platelet forms can also be found in U.S. Pat. No. 11,608,275, the entire contents of which is hereby incorporated by reference in its entirety.

Suitable examples of other coated oxides include, but are not limited to, coated oxides preferably having amphiphilic properties such as:
titanium oxides coated with cetyl phosphate and with silica, such as the product “Eusolex® T-EASY” from Merck;
titanium oxides coated with polyglyceryl-10 oleate and with stearic acid, such as “MTY-200STW” from Tayca;
zinc oxides coated with polyglyceryl-10 oleate and with isostearic acid, such as “MZY-505EXW” from Tayca;
titanium oxides coated with polysorbate 80 and with isostearic acid, such as “MT-10EXW” from Tayca; and
zinc oxides coated with polysorbate 80 and with isostearic acid, such as “MZY-304EXW” from Tayca.

Preferably, the at least one physical ultraviolet (UV)-attenuating material is/are present in compositions of the present disclosure in an amount of at least about 5% by weight, preferably at least about 10% by weight, preferably at least about 12% by weight, preferably at least about 14% by weight, and preferably at least about 15% by weight, with the upper end of the range of the at least one physical ultraviolet (UV)-attenuating material present preferably being about 40% by weight (e.g., about 5-40%, about 10-40%, about 12-40%, etc.), preferably about 30% by weight (e.g., about 5-30%, about 10-30%, about 12-30%, etc.), preferably about 25% by weight (e.g., about 5-25%, about 10-25%, about 12-25%, etc.), and preferably about 20% by weight (e.g., about 5-20%, about 10-20%, about 12-20%, etc.), with all weights being based on the total weight of the composition.

According to preferred embodiments, compositions of the present disclosure contain a UV absorbing system essentially containing physical ultraviolet (UV)-attenuating material(s) as defined above.

### Emollient System

According to the present disclosure, compositions comprising an emollient system comprising at least three emollients, wherein at least one emollient is a fast spreading emollient, at least one emollient is a medium spreading emollient, and at least one emollient is a slow spreading emollient, are provided.

The table below describes some emollients and their designation as fast spreading, medium spreading, or slow spreading according to their molecular weight.

| US INCI | MW (g/mol) | Spreading |
|---|---|---|
| Phenethyl benzoate | 226.3 | Fast |
| C15-19 alkane | 197 to about 249.4 | Fast |
| Isohexadecane | 226.4 | Fast |
| Diisopropyl adipate | 230.3 | Fast |
| Dicaprylyl ether | 242.4 | Fast |
| Isopropyl myristate | 270.5 | Medium |
| Isononyl isononanoate | 284.5 | Medium |
| Diisopropyl sebacate | 286.4 | Medium |
| Dicaprylyl carbonate | 286.4 | Medium |
| C12-15 alkyl benzoate | 290.4 | Medium |
| Octyl dodecanol | 298.5 | Medium |
| Isopropyl palmitate | 298.5 | Medium |
| Coco caprylate/caprate | About 312 | Slow |
| Isopropyl lauroyl | 313.5 | Slow |
| sarcosinate | | |
| Coco glycerides | About 320 | Slow |
| Ethylhexyl palmitate | 368.6 | Slow |
| Caprylic/capric | 380 | Slow |
| triglyceride | | |
| Cetearyl isononanoate | 382.7 | Slow |
| Dicaprylate/dicaprate | 380 | Slow |

Preferred fast spreading emollients include, but are not limited to, C15-19 alkane, dicaprylyl ether, isohexadecane, and mixtures thereof.

Preferred medium spreading emollients include, but are not limited to, dicaprylyl carbonate, diisopropyl sebacate, and mixtures thereof.

Preferred slow spreading emollients include, but are not limited to, cetearyl isononanoate.

In preferred embodiments of the present disclosure, the at least one fast spreading emollient, the at least one medium spreading emollient, and/or the at least one slow spreading emollient are selected from the group consisting of hydrocarbons, ethers, amides, esters, and mixtures thereof, each optionally containing one or more functional groups such as, for example, alcohols, thiols, amines, ketones, alkenes, and/or aromatic rings. The at least one fast spreading emollient, the at least one medium spreading emollient, and the at least one slow spreading emollient may comprise branched structures.

According to preferred embodiments, the emollient system can comprise, consist essentially of, or consist of hydrocarbons.

According to preferred embodiments, the emollient system is “free of,” “substantially free of,” or “devoid of” as defined above of silicones.

Preferably, the emollient system is present in compositions of the present disclosure in an amount of at least about 5% by weight, preferably at least about 10% by weight, preferably at least about 12% by weight, preferably at least about 14% by weight, and preferably at least about 15% by weight, with the upper end of the range of the emollient system present preferably being about 40% by weight (e.g., about 5-40%, about 10-40%, about 12-40%, etc.), preferably about 30% by weight (e.g., about 5-30%, about 10-30%, about 12-30%, etc.), preferably about 25% by weight (e.g., about 5-25%, about 10-25%, about 12-25%, etc.), and preferably about 20% by weight (e.g., about 5-20%, about 10-20%, about 12-20%, etc.), with all weights being based on the total weight of the composition.

Preferably, more fast spreading emollient(s) is/are present in compositions of the present disclosure than (1) medium spreading emollient(s) and/or (2) slow spreading emollient(s) by weight with respect to the total weight of the composition.

Preferably, more medium spreading emollient(s) is/are present in compositions of the present disclosure than slow spreading emollient(s) by weight with respect to the total weight of the composition.

Preferably, at least two times as much fast spreading emollient(s) is/are present in compositions of the present disclosure than medium spreading emollient(s) by weight with respect to the total weight of the composition, preferably at least 2.3 times as much, preferably at least 2.5 times as much, and preferably at least 3 times as much.

Preferably, at least two times as much medium spreading emollient(s) is/are present in compositions of the present disclosure than slow spreading emollient(s) by weight with respect to the total weight of the composition, preferably at least 2.3 times as much, preferably at least 2.5 times as much, and preferably at least 3 times as much.

Preferably, at least three times as much fast spreading emollient(s) is/are present in compositions of the present disclosure than medium spreading emollient(s) by weight with respect to the total weight of the composition, preferably at least 3.5 times as much, preferably at least 4 times as much, and preferably at least 5 times as much.

Preferably, the weight ratio of (1) the combined amount of the at least one fast spreading emollient, the at least one medium spreading emollient, and the at least one slow spreading emollient to (2) the at least one physical ultraviolet (UV)-attenuating material present in compositions of the present disclosure is from about 5:1 to about 1:5, preferably from about 3:1 to about 1:3, preferably from about 2:1 to about 1:2, and preferably from about 1.5:1 to about 1:1.5, including all ranges and subranges therebetween.

Without wishing to be bound by any theory, it is believed that combining emollients with the different, defined spreading characteristics with at least one physical ultraviolet (UV)-attenuating material allows for a sunscreen composition having a pleasing aesthetic throughout application of the sunscreen composition to keratinous material, while also inhibiting the white cast effect.

### Additional Sunscreen Agents

According to preferred embodiments of the present disclosure, compositions optionally further comprising at least one additional UV filter (in addition to at least one physical ultraviolet (UV)-attenuating material) selected from the group consisting of organic UV filters are provided. However, as noted above, preferred embodiments of the present disclosure include compositions of the present disclosure containing a UV absorbing system containing little or no organic UV filter as defined above.

Additional organic UV filter(s) can be hydrophilic or lipophilic. “Hydrophilic organic UV filter” means a water-soluble organic UV filter or a water-dispersible (in colloidal form) organic UV filter. “Lipophilic organic UV filter” means a UV filter which is dissolved or dispersed in colloidal form in a liquid fatty phase.

Suitable organic UV filters can be selected from the following non-exhaustive list of compounds: cinnamic compounds; anthranilate compounds; Para-aminobenzoic acid compounds; salicylic compounds; dibenzoylmethane compounds; camphor compounds; benzophenone compounds; β,β-diphenylacrylate compounds; triazine compounds such as Bis-Ethylhexyloxyphenol Methoxyphenyl Triazine; benzotriazole compounds; benzalmalonate compounds including those mentioned in patent U.S. Pat. No. 5,624,663; benzimidazole derivatives; imidazoline compounds; bis-benzoazolyl compounds as described in patents EP669323 and U.S. Pat. No. 2,463,264; methylene bis-(hydroxyphenyl benzotriazole) compounds as described in applications U.S. Pat. Nos. 5,237,071, 5,166,355, GB2303549, DE 197 26 184 and EP893119; benzoxazole compounds as described in patent applications EP0832642, EP1027883, EP1300137 and DE10162844; polymer filters and silicone filters such as those described in particular in application WO-93/04665; dimers derived from -alkylstyrene such as those described in patent application DE19855649; 4,4-diarylbutadienes compounds as described in applications EP0967200, DE19746654, DE19755649, EP-A-1008586, EP1133980 and EP133981 and mixtures thereof. Preferably, the lipophilic organic UV filters are selected from salicylic compounds, dibenzoylmethane compounds, benzylidene camphor compounds; benzophenone compounds; triazine compounds; benzotriazole compounds; as well as other categories of compounds identified herein; and mixtures thereof.

Specific reference can be made to suitable salicylic compounds including Homosalate (homomentyl salicylate), for example marketed under the trademark “Eusolex HMS” by Rona/EM Industries; and ethylhexyl salicylate, for example marketed under the trademark “Neo Heliopan OS” by Symrise; and glycol salicylate. Other examples of salicylate compounds include phenyl salicylate; dipropyleneglycol salicylate, for example marketed under the trademark “Dipsal” by Scher; and TEA salicylate, for example marketed under the trademark “Neo Heliopan TS” by Symrise.

Examples of suitable β,β-Diphenylacrylate compounds include Octocrylene, for example marketed under the trademark “Uvinul N539” by BASF; and Etocrylene, for example marketed under the trademark “Uvinul N35” by BASF.

Suitable anthranilic compounds can include menthyl anthranilates, for example marketed under the trademark “Neo Heliopan MA” by Symrise.

Examples of dibenzoylmethane compounds include Butyl methoxydibenzoylmethane, for example marketed under the trademark “Parsol 1789” by DSM; and isopropyl dibenzoylmethane.

Suitable cinnamic compounds include Ethylhexyl methoxycinnamate, for example marketed under the trademark “Parsol MCX” by DSM; isopropyl methoxycinnamate; isopropoxy methoxycinnamate; isoamyl methoxycinnamate, for example marketed under the trademark “Neo Heliopan E 1000” by Symrise; cinoxate (2-ethoxyethyl-4-methoxy cinnamate); DEA methoxycinnamate; diisopropyl methylcinnamate; and glyceryl ethylhexanoate dimethoxycinnamate.

Examples of camphor compounds include benzylidenecamphor derivatives: 3-benzylidene camphor, for example marketed under the trademark “Mexoryl SD” by Chimex; 4-methylbenzylidene camphor, for example marketed under the trademark “Eusolex 6300” by Merck; benzylidene camphor sulfonic acid, for example marketed under the trademark “Mexoryl SL” by Noveal; camphor benzalkonium methosulfate, for example marketed under the trademark “Mexoryl SO” by Noveal; terephthalylidene dicamphor sulfonic acid, for example marketed under the trademark “Mexoryl SX” by Noveal; and polyacrylamidomethyl benzylidene camphor, for example marketed under the trademark “Mexoryl SW” by Noveal.

Suitable benzophenone compounds include benzophenone-1 (2,4-dihydroxybenzophenone), such as that marketed under the trademark “Uvinul 400” by BASF; benzophenone-2 (Tetrahydroxybenzophenone), such as that marketed under the trademark “Uvinul D50” by BASF; Benzophenone-3 (2-hydroxy-4-methoxybenzophenone) or oxybenzone, such as that marketed under the trademark “Uvinul M40” by BASF; benzophenone-4 (hydroxymethoxy benzophonene sulfonic acid), such as that marketed under the trademark “Uvinul MS40” by BASF; benzophenone-5 (Sodium hydroxymethoxy benzophenone Sulfonate); benzophenone-6 (dihydroxy dimethoxy benzophenone); such as that marketed under the trademark “Helisorb 11” by Norquay; benzophenone-8, such as that marketed under the trademark “Spectra-Sorb UV-24” by American Cyanamid; benzophenone-9 (Disodium dihydroxy dimethoxy benzophenonedisulfonate), such as that marketed under the trademark “Uvinul DS-49” by BASF; and benzophenone-12, and n-hexyl 2-(4-diethylamino-2-hydroxybenzoyl)benzoate (such as that marketed under the tradename UVINUL A+ by BASF).

Examples of triazine compounds include 4-bis-{[4-(2-ethyl hexyloxy)-2-hydroxy]-phenyl]-6-(4-methoxyphenyl)-1,3,5-triazine (INCI name: Bis-Ethylhexyloxyphenol Methoxyphenyl Triazine (BEMT)), Diethylhexyl butamido triazone, such as that marketed under the trademark “Uvasorb HEB” by Sigma 3V; 2,4,6-tris (dineopentyl 4′-aminobenzalmalonate)-s-triazine, bis-ethylhexyloxyphenol methoxyphenyl triazine, such as that marketed under the trademark “TINOSORB S” by BASF, and ethylhexyl triazone, such as that marketed under the trademark “UVTNUL T150” by BASF.

Suitable benzotriazole compounds include phenylbenzotriazole derivatives: 2-(2H-benzotriazole-2-yl)-6-dodecyl-4-methylpheno, branched and linear; and those described in U.S. Pat. No. 5,240,975.

Suitable benzalmalonate compounds include Dineopentyl 4′-methoxybenzalmalonate, and polyorganosiloxane comprising benzalmalonate functional groups, such as polysilicone-15, such as that marketed under the trademark “Parsol SLX” by Hoffmann-LaRoche.

Examples of benzimidazole compounds include, in particular, phenylbenzimidazole derivatives such as phenylbenzimidazole sulfonic acid, such as that marketed in particular under the trademark “Eusolex 232” by Merck, and disodium phenyl dibenzimidazole tetrasulfonate, such as that marketed under the trademark “Neo Heliopan AP” by Symrise.

Suitable imidazoline compounds include Ethylhexyl dimethoxybenzylidene dioxoimidazoline propionate.

Examples of bis-benzoazolyl compounds include the compounds described in EP-669.323 and U.S. Pat. No. 2,463,264.

Suitable para-aminobenzoic acid compounds include PABA (p-aminobenzoic acid), ethyl PABA, Ethyl dibydroxypropyl PABA, pentyl dimethyl PABA, ethylhexyl dimethyl PABA, such as that marketed under the trademark “Escalol 507” by ISP, glyceryl PABA, and PEG-25 PABA, such as that marketed under the trademark “Uvinul P25” by BASF.

Suitable methylene bis-(hydroxyphenylbenzotriazol) compounds include 2,2′-methylenebis [6-(2H-benzotriazol-2-yl)-4-methyl-phenol], such as that marketed under the trademark “Mixxim BB/200” by Fairmount Chemical, 2,2′-methylenebis [6-(2H-benzotriazol-2-yl)-4-(1,1,3,3-tetramethylbutyl) phenol], such as that marketed in the micronized form in aqueous dispersion under the trademark “Tinosorb M” by BASF, or under the trademark “Mixxim BB/100” by Fairmount Chemical, and the derivatives as described in U.S. Pat. Nos. 5,237,071 and 5,166,355, GB-2,303,549, DE-197,26,184, and EP-893,119, and Drometrizole trisiloxane, such as that marketed under the trademark “Silatrizole” by Rhodia Chimie or- “Mexoryl XL” by L′Oréal.

Examples of benzoxazole compounds include 2,4-bis [5-1 (dimethylpropyl)benzoxazol-2-yl-(4-phenyl) imino]-6-(2-ethylhexyl) imino-1,3,5-triazine, such as that marketed under the trademark of Uvasorb K2A by Sigma 3V.

Suitable examples of screening polymers and screening silicones include the silicones described in WO 93/04665.

Suitable dimers derived from a-alkylstyrene include the dimers described in DE-19855649.

Examples of 4,4-Diarylbutadiene compounds include 1,1-dicarboxy (2,2′-dimethylpropyl)-4,4-diphenylbutadiene.

If present, the at least one additional organic UV filter is preferably present in compositions of the present disclosure in an amount of at least about 1% by weight, preferably at least about 5% by weight, preferably at least about 10% by weight, preferably at least about 12.5% by weight, and preferably at least about 15% by weight, with the upper end of the range of additional UV filter present preferably being about 40% by weight (e.g., about 1-40%, about 10-40%, about 12.5-40%, etc.), preferably about 30% by weight (e.g., about 5-30%, about 10-30%, about 15-30%, etc.), preferably about 25% by weight (e.g., about 5-25%, about 10-25%, about 15-25%, etc.), and preferably about 20% by weight (e.g., about 1-20%, about 5-20%, about 10-20%, etc.), with all weights being based on the total weight of the composition.

According to preferred embodiments, compositions of the present disclosure comprise 10% or less by weight relative to the total weight of composition of such optional additional UV filters, preferably less than 7.5% by weight relative to the total weight of composition, preferably less than 5% by weight relative to the total weight of composition, preferably less than 3% by weight relative to the total weight of composition, and preferably less than 1% by weight relative to the total weight of composition.

According to preferred embodiments, compositions of the present disclosure further comprise at least one additional organic UV filter selected from the group consisting of 4-bis-{[4-(2-ethyl hexyloxy)-2-hydroxy]-phenyl]-6-(4-methoxyphenyl)-1,3,5-triazine (INCI name: Bis-Ethylhexyloxyphenol Methoxyphenyl Triazine), Avobenzone (butyl methoxydibenzoylmethane), Octisalate (ethylhexyl salicylate), Ensulizole (Phenylbenzimidazole sulfonic acid), Homosalate, Octocrylene, and mixtures thereof. In such embodiments, the UV absorbing system can “consist of” or “consist essentially of” (1) at least one physical ultraviolet (UV)-attenuating material and (2) at least one organic UV filter selected from the group consisting of 4-bis-{[4-(2-ethyl hexyloxy)-2-hydroxy]-phenyl]-6-(4-methoxyphenyl)-1,3,5-triazine (INCI name: Bis-Ethylhexyloxyphenol Methoxyphenyl Triazine), Avobenzone (butyl methoxydibenzoylmethane), Octisalate (ethylhexyl salicylate), Ensulizole (Phenylbenzimidazole sulfonic acid), Homosalate, Octocrylene, and mixtures thereof.

According to other preferred embodiments, however, compositions of the present disclosure are “free of,” “substantially free of,” or “devoid of” as defined above of one or more of additional organic UV filters selected from the group consisting of 4-bis-{[4-(2-ethyl hexyloxy)-2-hydroxy]-phenyl]-6-(4-methoxyphenyl)-1,3,5-triazine (INCI name: Bis-Ethylhexyloxyphenol Methoxyphenyl Triazine), Avobenzone (butyl methoxydibenzoylmethane), Octisalate (ethylhexyl salicylate), Ensulizole (Phenylbenzimidazole sulfonic acid), Homosalate, and Octocrylene, preferably two or more, preferably three or more, preferably four or more, or preferably all five of these sunscreen agents.

According to preferred embodiments, compositions of the present disclosure are “free of,” “substantially free of,” or “devoid of” as defined above of one or more of additional organic UV filters selected from the group consisting of OXYBENZONE (benzophenone-3), OCTINOXATE (Ethylhexyl methoxycinnamate), ETHYLHEXYL TRIAZONE, DROMETRIZOLE TRISILOXANE, METHYLENE BIS-BENZOTRIAZOLYL TETRAMETHYLBUTYL PHENOL, DIETHYLAMINO HYDROXY BENZOYL HEXYL BENZOATE, DIETHYLHEXYL BUTAMIDO TRIAZONE, ISOAMYL P-METHOXYCINNAMATE, POLYSILICONE-15, 4-METHYLBENZYLIDENE CAMPHOR, DISODIUM PHENYL DIBENZIMIDAZOLE TETRASULFONATE, METHOXYPROPYLAMINO CYCLOHEXENYLIDENE ETHOXYETHYLCYANOACETATE, 4-bis-{[4-(2-ethyl hexyloxy)-2-hydroxy]-phenyl]-6-(4-methoxyphenyl)-1,3,5-triazine (INCI name: Bis-Ethylhexyloxyphenol Methoxyphenyl Triazine), preferably two or more, preferably three or more, preferably four or more, etc., and preferably “free of,” “substantially free of,” or “devoid of” all of these sunscreen agents.

According to preferred embodiments, compositions of the present disclosure are “free of,” “substantially free of,” or “devoid of” as defined above of OXYBENZONE (benzophenone-3) and/or OCTINOXATE (Ethylhexyl methoxycinnamate) and/or 4-bis-{[4-(2-ethyl hexyloxy)-2-hydroxy]-phenyl]-6-(4-methoxyphenyl)-1,3,5-triazine (INCI name: Bis-Ethylhexyloxyphenol Methoxyphenyl Triazine).

According to preferred embodiments, the UV absorbing system of compositions of the present disclosure can “consist of” or “consist essentially of” at least one physical ultraviolet (UV)-attenuating material.

According to preferred embodiments, this disclosure contemplates omitting one or more of any of the specific UV filters discussed above from the UV absorbing system of compositions of the present disclosure. By way of example, octocrylene and/or octinoxate can be omitted from the compositions. Similar omission of one or more of any of the specific UV filters discussed is thus contemplated.

### Antioxidants

According to preferred embodiments of the present disclosure, compositions of the present disclosure can optionally further comprise at least one antioxidant. Examples of such antioxidants include, but are not limited to, vitamins, ascorbic acid, polyphenols, flavonoids, stilbenoids, and derivatives thereof. Antioxidants may be dissolved in either the oily or aqueous phases.

Suitable specific examples of antioxidants include, but are not limited to, flavones, flavonoids, flavans, flavanones, flavonols, stilbenoids, ascorbic acid (Vitamin C), ascorbyl glucoside, ascorbyl palmitate, tocopherol (Vitamin E), tocopheryl acetate, (octyl) Methoxycrylene (SolaStay S1), Diethylhexyl 2,6-Naphthalate (DEHN) (Corapan TQ), Phenylethyl Benzoate, Butyloctyl Salicylate (HallBrite BHB), Diethylhexyl Syringylidene Malonate (Oxynex ST), Polycrylene (Polyester-8), Hexadecyl Benzoate, Butyloctyl Benzoate, and mixtures thereof.

### Aqueous Phase

The compositions of the present disclosure may also optionally contain water. When the compositions of the present disclosure contain water, they are preferably in the form of an emulsion. Preferably, when the compositions of the present disclosure contain water, they are in the form of an emulsion containing an external aqueous phase such as an oil-in-water emulsion (O/W) or a water-in-oil-in-water emulsion (O/W/O), or an emulsion containing an external oil phase such as a water-in-oil emulsion (W/O) or an oil-in-water-in-oil (O/W/O) emulsion. Preferably, when in the form of an emulsion, the oil phase can contain silicone oils (e.g., Si/W or W/Si emulsion) or hydrocarbon oils. When present, water is preferably present in an amount of from about 10% to about 80% by weight, preferably from about 20% to about 70% by weight, preferably from about 35% to about 65% by weight, including all ranges and subranges therebetween, all weights being based on the total weight of the composition.

According to preferred embodiments, however, compositions of the present disclosure are devoid of water, substantially free of water, or free of water as defined herein. Preferably, the compositions of the present disclosure are anhydrous.

If present in compositions of the present disclosure, the aqueous phase may comprise at least one water-soluble organic solvent which is liquid at room temperature and atmospheric pressure. For example, such at least one water-soluble organic solvent may include:
C1-C5 monoalcohols having a C1-C5 alkane chain and a single hydroxyl function (OH). Suitable C1-C5 monoalcohols include methanol, ethanol, propanol, isopropanol, butanol and mixtures thereof;
polyols (compounds having 2 or more hydroxyl groups) having, for example from 2 to 20 carbon atoms, preferably from 2 to 6 carbon atoms, such as for example glycerol, diglycerol, propylene glycol, isoprene glycol, dipropylene glycol, butylene glycol, hexylene glycol, 1,3-propanediol, pentylene glycol, simple sugars, and water-soluble polyalkylene glycols;
and mixtures thereof.

According to preferred embodiments, the at least one water-soluble organic solvent is selected from the group consisting of ethanol, dipropylene glycol, butylene glycol, propanediol and propylene glycol, and mixtures thereof.

If present, the water-soluble organic solvent(s) is/are preferably present in compositions of the present disclosure in an amount ranging from about 0.5 to about 40% by weight, preferably from about 3 to about 30% by weight, and preferably from about 5% to about 20% by weight relative to the total weight of the composition, including all ranges and subranges therebetween such as, for example, 2% to 15%, 2% to 25%, 7.5% to 30%, etc.

### Additional Optional Ingredients

Compositions of the present disclosure may also optionally further include at least one additive or auxiliary commonly used in cosmetic compositions and known to a person skilled in the art as being capable of being incorporated into such compositions. Such additives or auxiliaries may be chosen from film formers, coloring agents (e.g., dyes and pigments), waxes, thixotropic agents (e.g., clays), fillers, preservatives, fragrances, surfactants, antioxidants, agents for combating free radicals, spreading agents, dispersing agents, antifoaming agents, neutralizing agents, stabilizing agents, active principles chosen from essential oils, moisturizing agents, vitamins, actives, proteins, ceramides, plant extracts, fibers, and the like, wetting agents and their mixtures. Although, preferably, compositions of the present disclosure are “free of,” “substantially free of,” or “devoid of” such additives.

A person skilled in the art will take care to select the optional additional additives and/or the amount thereof such that the advantageous properties of the composition according to the disclosure are not, or are not substantially, adversely affected by the envisaged addition.

Needless to say, the composition of the disclosure should be cosmetically or dermatologically acceptable, i.e., it should contain a non-toxic physiologically acceptable. The composition may be in any galenic form normally employed in the cosmetic and dermatological fields which is suitable for topical administration as discussed above.

These auxiliary additives may be present in the composition in a proportion from 0% to 99% (such as from 0.01% to 90%) relative to the total weight of the composition and further such as from 0.1% to 50% (if present), including all ranges and subranges therebetween.

In accordance with the present disclosure, compositions of the present disclosure can be a stand-alone product (for use by itself), or they can be a product for use in conjunction with another composition, for example it can be a basecoat (primer) composition, a color coat composition, or a topcoat (over coat) composition. It should be understood that when compositions of the present disclosure are applied to keratinous materials in the form of any of such compositions, such application can comprise one or more layers of the product. Thus, for example, application of at least one color coat composition can comprise one or more color coat layers; application of the at least topcoat composition can comprise one or more topcoat layers; and application of the at least one basecoat composition can comprise one or more basecoat layers. Preferably, such basecoat, color coat and topcoat compositions contain three or fewer layers of compositions, preferably two or fewer layers of compositions, and preferably a single layer of compositions.

During application of compositions of the present disclosure, basecoat (if present) is typically applied directly to keratinous material, color coat is typically applied either directly to the keratinous material (if no basecoat is present) or to a previously applied basecoat, and topcoat (if present) is typically applied to a color coat.

According to preferred embodiments of the present disclosure, methods of treating, protecting, enhancing the appearance of, caring for and/or making up keratinous material by applying compositions of the present disclosure to the keratinous material in an amount sufficient to treat, enhance the appearance of, care for and/or make up the keratinous material are provided.

Preferably, “making up” keratinous material includes applying a composition comprising at least one coloring agent to the keratinous material in an amount sufficient to provide color and/or optical effect to the keratinous material.

Preferably, “protecting” keratinous material includes applying a composition of the present disclosure to protect keratinous material from damage resulting from exposure to UV rays.

In accordance with the preceding embodiments, compositions of the present disclosure are applied topically to the keratinous material in an amount sufficient to treat, enhance the appearance of, care for and/or make up the keratinous material. The compositions may be applied to the desired area as needed, preferably once or twice daily, more preferably once daily and then preferably allowed to dry before subjecting to contact such as with clothing or other objects (for example, clothes or a topcoat). Preferably, the composition is allowed to dry for about 1 minute or less, more preferably for about 45 seconds or less.

According to preferred embodiments of the present disclosure, methods of making non-greasy and/or non-whitening compositions comprising at least one physical ultraviolet (UV)-attenuating material by combining at least one physical ultraviolet (UV)-attenuating material, at least one fast spreading emollient, at least one medium spreading emollient, and at least one slow spreading emollient in the compositions during formation of the compositions to produce compositions which are non-greasy and/or non-whitening are provided.

According to preferred embodiments of the present disclosure, methods of improving sensory properties upon application to skin of a composition comprising at least one physical ultraviolet (UV)-attenuating material, wherein the method comprises adding at least one fast spreading emollient, at least medium spreading emollient, and at least one slow spreading emollient to a composition comprising at least one physical ultraviolet (UV)-attenuating material during formation of the composition in amounts sufficient to improve sensory properties of the composition upon application to skin are provided.

According to all embodiments of the present disclosure, it is preferred that more fast spreading emollient(s) is/are added into compositions of the present disclosure during preparation than (1) medium spreading emollient(s) and/or (2) slow spreading emollient(s) by weight with respect to the total weight of the composition.

Also preferably, more medium spreading emollient(s) is/are added into compositions of the present disclosure during preparation than slow spreading emollient(s) by weight with respect to the total weight of the composition.

Also preferably, at least two times as much fast spreading emollient(s) is/are added into compositions of the present disclosure during preparation than medium spreading emollient(s) by weight with respect to the total weight of the composition, preferably at least 2.3 times as much, preferably at least 2.5 times as much, and preferably at least 3 times as much.

Also preferably, at least two times as much medium spreading emollient(s) is/are added into compositions of the present disclosure during preparation than slow spreading emollient(s) by weight with respect to the total weight of the composition, preferably at least 2.3 times as much, preferably at least 2.5 times as much, and preferably at least 3 times as much.

Also preferably, at least three times as much fast spreading emollient(s) is/are added into compositions of the present disclosure during preparation than medium spreading emollient(s) by weight with respect to the total weight of the composition, preferably at least 3.5 times as much, preferably at least 4 times as much, and preferably at least 5 times as much.

Also preferably, the weight ratio of (1) the combined amount of the at least one fast spreading emollient, the at least one medium spreading emollient, and the at least one slow spreading emollient to (2) the at least one physical ultraviolet (UV)-attenuating material added into compositions of the present disclosure during preparation is from about 5:1 to about 1:5, preferably from about 3:1 to about 1:3, preferably from about 2:1 to about 1:2, and preferably from about 1.5:1 to about 1:1.5, including all ranges and subranges therebetween.

The present disclosure also envisages kits and/or prepackaged materials suitable for consumer use containing one or more compositions according to the description herein, alone or in combination with other consumer care products such as makeup products such as basecoats, topcoats, removal compositions, etc. The packaging and application device for any subject of the disclosure may be chosen and manufactured by persons skilled in the art on the basis of their general knowledge, and adapted according to the nature of the composition to be packaged. Indeed, the type of device to be used can be in particular linked to the consistency of the composition, in particular to its viscosity; it can also depend on the nature of the constituents present in the composition, such as the presence of volatile compounds.

Particularly preferred embodiments include:
A composition comprising a UV absorbing system comprising at least one physical ultraviolet (UV)-attenuating material, preferably at least one metal oxide, preferably at least one metal oxide selected from oxides of titanium, chromium, zinc, tin, alumina, cerium, and/or iron; and an emollient system comprising at least three emollients, wherein at least one emollient is at least one fast spreading emollient, at least one emollient is at least one medium spreading emollient, and at least one emollient is at least one slow spreading emollient;
The composition of the preceding embodiment, wherein (1) the at least one fast spreading emollient is selected from the group consisting of C15-19 alkane, isohexadecane, dicaprylyl ether, and mixtures thereof. (2) the at least one medium spreading emollient is selected from the group consisting of dicaprylyl carbonate, diisopropyl sebacate, and mixtures thereof, and/or (3) least one slow spreading emollient is cetearyl isononanoate;
The composition of any preceding embodiment, wherein the at least one physical ultraviolet (UV)-attenuating material is present in the composition in an amount of at least 5% by weight with respect to the total weight of the composition, preferably about 5% to about 40% by weight with respect to the total weight of the composition;
The composition of any preceding embodiment, wherein the at least one fast spreading emollient, the at least one medium spreading emollient, and the at least one slow spreading emollient are present in the composition in a combined amount of at least 5% by weight with respect to the total weight of the composition, preferably about 5% to about 40% by weight with respect to the total weight of the composition;
The composition of any preceding embodiment, wherein the composition is devoid of organic UV filters;
The composition of any preceding embodiment, wherein the composition is non-greasy and/or non-whitening upon application to keratinous material;
The composition of any preceding embodiment, in the form of an emulsion;
The composition of any preceding embodiment, wherein more fast spreading emollient(s) is/are present in the composition by weight with respect to the total weight of the composition than (1) medium spreading emollient(s) and/or (2) slow spreading emollient(s);
The composition of any preceding embodiment, wherein more medium spreading emollient(s) is/are present in the composition by weight with respect to the total weight of the composition than slow spreading emollient(s);
The composition of any preceding embodiment, wherein the at least one UV absorbing system essentially contains at least one physical ultraviolet (UV)-attenuating material;
The composition of any preceding embodiment, wherein the at least one physical ultraviolet (UV)-attenuating material is selected from the group consisting of titanium dioxide, zinc oxide, iron oxide, chromium oxide, tin oxide, alumina, cerium oxide, and mixtures thereof;
T The composition of any preceding embodiment, wherein the at least one physical ultraviolet (UV)-attenuating material is surface treated with a surface treatment agent, preferably the surface treating agent comprising at least one of amino acids, beeswax, fatty acids, fatty acid salts, fatty alcohols, anionic surfactants, lecithin compounds, metal alkoxides, polyethylene, silicones, proteins, alkanolamines, silicon oxides, metal oxides different from the at least one physical ultraviolet (UV)-attenuating material, sodium hexametaphosphate, alumina, glycerol, and mixtures thereof;
The composition of any preceding embodiment, wherein the at least one physical ultraviolet (UV)-attenuating material comprises zinc oxide and/or titanium dioxide, preferably passivated;
The composition of any preceding embodiment, wherein the at least one physical ultraviolet (UV)-attenuating material comprises zinc oxide and/or titanium dioxide, preferably coated with a coating agent, preferably the coating agent comprising at least one of hydrated silica, triethoxysilylethyl polydimethylsiloxyethyl hexyl dimethicone, hydrogen dimethicone, triethoxycaprylylsilane, and mixtures thereof;
The composition of any preceding embodiment, wherein the at least one physical ultraviolet (UV)-attenuating material comprises zinc oxide, preferably passivated or coated with hydrated silica and/or hydrogen dimethicone;
The composition of any preceding embodiment, wherein the at least one physical ultraviolet (UV)-attenuating material has a mean primary particle size of from 1 nm to 500 nm, preferably from 5 nm to 250 nm, preferably from 10 nm to 100 nm, preferably from 20 nm to 50 nm; and
The composition of any preceding embodiment, wherein the at least one physical ultraviolet (UV)-attenuating material has a mean primary particle size of about 10 nm to about 40 nm.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective measurements. The following examples are intended to illustrate the disclosure without limiting the scope as a result. The percentages are given on a weight basis.

### Example 1—Sample Formulations

| | General Ranges | Preferred Ranges |
|---|---|---|
| INCI Name | (wt %) | (wt %) |
| Oils including | Total oil = 5-30% | Total oil = 10-25% |
| specified fast, medium | Including: | Including: |
| and slow spreading | Cetearyl isononanoate | Cetearyl isononanoate |
| emollients | (S) (1-5%) | (S) (2-4%) |
| | Dicaprylyl carbonate | Dicaprylyl carbonate |
| | (M) (3-8%) | (M) (4-6%) |
| | Isohexadecane (F) | Isohexadecane (F) |
| | (4-12%) | (5-10%) |
| | C15-19 alkane (F) | C15-19 alkane (F) |
| | (1-5%) | (1-5%) |
| Surfactants | 0.5-7.5% | 1-5% |
| Thickeners/Film | 0.5-5% | 1-4% |
| Formers | | |
| Zinc Oxide (alumina, | 15-40% | 10-20% |
| polyhydroxystearic | | |
| acid coating) | | |
| Titanium Dioxide | 1-10% | 1-5% |
| Filler | 0.5-10% | 1-5% |
| Polyol | 0-20% | 1-10% |
| Other Cosmetic | 0-25% | 5-20% |
| Ingredients | | |
| (preservatives, | | |
| chelators, | | |
| antioxidants, organic | | |
| sunscreen agents) | | |
| Water | QS | QS |

### Example 2—Transparency Testing

Mixtures containing 15 wt % zinc oxide, treated with triethoxycaprylylsilane, and 2 wt % polyhydroxystearic acid dispersed in different emollients were prepared as identified in Table 2A below. The mixtures were prepared by mixing under high shear at high temperature (above 50 degrees Celsius) until the zinc oxide material was well-dispersed. Transparency of the mixtures in Table 2A was assessed by comparing the absorbance values at 650 nm (turbidity). To measure turbidity (absorbance at 650 nm), 21 microliters of the mixture was diluted to 210 microliters with emollient, then measured in a 96-well flat-bottomed polyacrylate plate using a spectrophotometer. Lower absorbance values corresponded to higher transparency and are used herein to establish the relative transparency among mixtures prepared according to Table 2A.

TABLE 2A
Mixtures of emollient and 15 wt % zinc
oxide (and) triethoxycaprylylsilane

| Emollient | Spreading | Absorbance at 650 nm |
|---|---|---|
| Phenethyl benzoate | Fast | 2.204 |
| C15-19 alkane | Fast | 2.7565 |
| Isohexadecane | Fast | 2.8177 |
| Diisopropyl adipate | Fast | 3.2259 |
| Dicaprylyl ether | Fast | 2.6921 |
| Isopropyl myristate | Medium | 2.701 |
| Isononyl isononanoate | Medium | 2.7417 |
| Diisopropyl sebacate | Medium | 2.6696 |
| Dicaprylyl carbonate | Medium | 2.8189 |
| C12-15 alkyl benzoate | Medium | 2.4816 |
| Octyl dodecanol | Medium | 2.3077 |
| Ethylhexyl palmitate | Slow | 2.5365 |
| Coco caprylate/caprate | Slow | 2.695 |
| Isopropyl lauroyl sarcosinate | Slow | 2.6783 |
| Coco glycerides | Slow | 2.5554 |
| Caprylic/capric triglyceride | Slow | 2.2453 |
| Cetearyl isononanoate | Slow | 2.6164 |
| Dicaprylate/dicaprate | Slow | 2.7063 |

The mixtures of different emollient and zinc oxide (and) tricthoxycaprylylsilane yielded different absorbance values at 650 nm as reflected in Table 2A, demonstrating that transparency values are affected by choice of emollient in the presence of mineral filter (exemplified by zinc oxide).

### Example 3—Transparency and Sensory Testing of Sample Oil-In-Water Formulations Containing Zinc Oxide

TABLE 3A

| INCI Name | Wt % |
|---|---|
| Emollients | 30 |
| Steareth-20 | 1 |
| Steareth-2 | 0.6 |
| Stearyl Alcohol | 0.6 |
| Glyceryl Stearate | 0.7 |
| Polysobate 20 | 0.7 |
| Water | Q.S. |
| Hydroxyethyl Acrylate/Sodium | 0.43 |
| Acryloyldimethyl Taurate Copolymer | |
| Preservative | 0.7 |
| Zinc Oxide (and) | 7.15 |
| triethoxycaprylylsilane | |

Oil-in-water compositions in the above Table 3A were prepared according to the methods in Example 2 above. Different emollients were used in different formulations. Zinc oxide was dispersed under high shear in each emollient and combined with the other ingredients under high shear for 20 min at or around 75 C until well-dispersed.

Sensory properties of the formulas prepared were compared by dispensing approximately 0.5 g of each formula to the back of a cleaned hand and then consistently rubbing each dispensed formula with the opposing hand. Sensory properties were scored overall on a scale of from 1 (preferable sensory) to 5 (unpleasant sensory). Sensory characteristics which were preferable included being easy to spread, having a lightweight feel on skin, feeling mild or no tackiness, not being too thin or runny (pleasant consistency), and not leaving significant residue on fingers or a residue feel on skin. Sensory characteristics which were unpleasant included being greasy or draggy (difficult to spread), being tacky or sticky, having a feeling of residue on fingers or on skin, or possessing an unpleasant consistency.

Transparency of the compositions was assessed by comparing the absorbance values at 650 nm (turbidity). To measure turbidity (absorbance at 650 nm), 21 microliters of the composition was diluted to 210 microliters with water, then measured in a 96-well flat-bottomed polyacrylate plate using a spectrophotometer. Lower absorbance values corresponded to a higher transparency and are used herein to establish the relative transparency among compositions prepared. Compositions were found to be suitably transparent when applied to skin when values of absorbance at 650 nm measured in the aforementioned manner were around or under 2.5.

Oil-in-water emulsions combining three different emollients and zinc oxide in Table 3B were thus prepared and tested:

TABLE 3B
Compositions with zinc oxide and different emollients

| | | | | Absor- | Sensory |
|---|---|---|---|---|---|
| Compo- | | | | bance at | Score |
| sition | Emollient 1 | Emollient 2 | Emollient 3 | 650 nm | (1-5) |
| Compar- | FAST | FAST | FAST | 2.4769 | 4 |
| ative 1 | (C15-19 | (diisopropyl | (dicaprylyl | | |
| | alkane) | adipate) | ether) | | |
| Compar- | MEDIUM | MEDIUM | MEDIUM | 2.545 | 3 |
| ative 2 | (isononyl | (dicaprylyl | (octyl | | |
| | isonon- | carbonate) | dodecanol) | | |
| | anoate) | | | | |
| Compar- | SLOW (coco | SLOW | SLOW | 2.3902 | 4 |
| ative 3 | glycerides) | (caprylic/ | (cetearyl | | |
| | | capric | isonon- | | |
| | | triglyceride) | anoate) | | |
| Compar- | MEDIUM | SLOW | SLOW | 2.6745 | 4 |
| ative 4 | (isononyl | (caprylic/ | (cetearyl | | |
| | isonon- | capric | isonon- | | |
| | anoate) | triglyceride) | anotate) | | |
| Compar- | FAST | FAST | SLOW | 3.0469 | 2 |
| ative 5 | (C15-19 | (dicaprylyl | (caprylic/ | | |
| | alkane) | ether) | capric | | |
| | | | triglyceride) | | |
| Compar- | FAST | MEDIUM | MEDIUM | 2.2322 | 2 |
| ative 6 | (isohexa- | (isopropyl | (octyl | | |
| | decane) | myristate) | dodecanol) | | |
| Inven- | FAST | MEDIUM | SLOW | 2.4558 | 1 |
| tive 1 | (C15-19 | (dicaprylyl | (cetearyl | | |
| | alkane) | carbonate) | isonon- | | |
| | | | anoate) | | |

The oil-in-water emulsions in Table 3B demonstrated the influence that emollient combinations have on transparency (absorbance at 650 nm) and sensory properties of compositions containing mineral filter (exemplified by zinc oxide). Mixtures of emollients which had the same spreading characteristics (Fast, medium, or slow) offered no sensory improvement. (Comparative exs. 1, 2, and 3). Compositions which combined emollients with different spreading characteristics generally demonstrated improved sensory characteristics as compared to compositions which combined emollients with the same spreading characteristics (compare comparative examples 4, 5, and 6 with comparative examples 1, 2, and 3, and inventive example 1). However, surprisingly, only inventive composition 1 in Table 3B demonstrated a combination of high transparency (2.4558) and the most preferred sensory score (1). Inventive composition 1 was found to have optimal slip without feeling draggy, felt lightweight on skin, did not leave a noticeable residue on fingers or on skin, and was appreciated for having a consistency which was pleasant and easy to apply.

Example 4 - Sample Sunscreen Composition

| Ingredient | Wt % |
|---|---|
| Zinc oxide (and) triethoxycaprylylsilane | 15.35% |
| Titanium dioxide (and) alumina (and) aluminum stearate | 2.65% |
| Polyhydroxy stearic acid | 2% |
| Surfactants | 3% |
| C15-19 alkane (fast spreading emollient) | 7% |
| Dicaprylyl carbonate (medium spreading emollient) | 5% |
| Cetearyl isononanoate (slow spreading emollient) | 2.5% |
| Additional oily phase ingredients | 5.1% |
| Aqueous thickening agents | 0.45% |
| Additional ingredients (preservatives, vitamins, pH | 15.4% |
| adjusters, aesthetic modifiers, glycols) | |
| Water | Q. S. |
| SPF vivo | 55 |
| Critical Wavelength | 372 |

The composition above was prepared and demonstrated a pleasant, lubricious application throughout application of the composition to keratinous skin without feeling heavy or greasy. Additionally, the composition did not show any visibly noticeable white effect after application.

What is claimed is:

1. A composition comprising a UV absorbing system comprising at least one physical ultraviolet (UV)-attenuating material, preferably at least one metal oxide, preferably at least one metal oxide selected from oxides of titanium, chromium, zinc, tin, alumina, cerium, and/or iron; and an emollient system comprising at least three emollients, wherein at least one emollient is at least one fast spreading emollient, at least one emollient is at least one medium spreading emollient, and at least one emollient is at least one slow spreading emollient.

2. The composition of claim 1, wherein (1) the at least one fast spreading emollient is selected from the group consisting of C15-19 alkane, isohexadecane, dicaprylyl ether, and mixtures thereof, (2) the at least one medium spreading emollient is selected from the group consisting of dicaprylyl carbonate, diisopropyl sebacate, and mixtures thereof, and/or (3) least one slow spreading emollient is cetearyl isononanoate.

3. The composition of claim 1, wherein the at least one physical ultraviolet (UV)-attenuating material is present in the composition in an amount of at least 5% by weight with respect to the total weight of the composition, preferably about 5% to about 40% by weight with respect to the total weight of the composition.

4. The composition of claim 1, wherein the at least one fast spreading emollient, the at least one medium spreading emollient, and the at least one slow spreading emollient are present in the composition in a combined amount of at least 5% by weight with respect to the total weight of the composition, preferably about 5% to about 40% by weight with respect to the total weight of the composition.

5. The composition of claim 1, wherein the composition is devoid of organic UV filters.

6. The composition of claim 1, wherein the composition is non-greasy and/or non-whitening upon application to keratinous material.

7. The composition of claim 1, in the form of an emulsion.

8. The composition of claim 1, wherein more fast spreading emollient(s) is/are present in the composition by weight with respect to the total weight of the composition than (1) medium spreading emollient(s) and/or (2) slow spreading emollient(s).

9. The composition of claim 1, wherein more medium spreading emollient(s) is/are present in the composition by weight with respect to the total weight of the composition than slow spreading emollient(s).

10. The composition of claim 1, wherein the at least one UV absorbing system essentially contains at least one physical ultraviolet (UV)-attenuating material.

11. The composition of claim 1, wherein the at least one physical ultraviolet (UV)-attenuating material is selected from the group consisting of titanium dioxide, zinc oxide, iron oxide, chromium oxide, tin oxide, alumina, cerium oxide, and mixtures thereof.

12. The composition of claim 1, wherein the at least one physical ultraviolet (UV)-attenuating material is surface treated with a surface treatment agent, preferably the surface treating agent comprising at least one of amino acids, beeswax, fatty acids, fatty acid salts, fatty alcohols, anionic surfactants, lecithin compounds, metal alkoxides, polyethylene, silicones, proteins, alkanolamines, silicon oxides, metal oxides different from the at least one physical ultraviolet (UV)-attenuating material, sodium hexametaphosphate, alumina, glycerol, and mixtures thereof.

13. The composition of claim 1, wherein the at least one physical ultraviolet (UV)-attenuating material comprises zinc oxide and/or titanium dioxide, preferably passivated.

14. The composition of claim 1, wherein the at least one physical ultraviolet (UV)-attenuating material comprises zinc oxide and/or titanium dioxide, preferably coated with a coating agent, preferably the coating agent comprising at least one of hydrated silica, triethoxysilylethyl polydimethylsiloxyethyl hexyl dimethicone, hydrogen dimethicone, triethoxycaprylylsilane, and mixtures thereof.

15. The composition of claim 1, wherein the at least one physical ultraviolet (UV)-attenuating material comprises zinc oxide, preferably passivated or coated with hydrated silica and/or hydrogen dimethicone.

16. The composition of claim 1, wherein the at least one physical ultraviolet (UV)-attenuating material has a mean primary particle size of from 1 om to 500 nm, preferably from 5 nm to 250 nm, preferably from 10 nm to 100 nm, preferably from 20 nm to 50 nm.

17. The composition of claim 1, wherein the at least one physical ultraviolet (UV)-attenuating material has a mean primary particle size of about 10 nm to about 40 nm.
